(19) United States
(12) Patent Application Publication
(10) Pub. No.: US 2026/0247062 A1
(43) Pub. Date: Aug. 20, 2026

(54) EXTERNAL AUDITORY CANAL MOUNTING DEVICE

(71) Applicant: J-PHONIC CO., LTD., Yamanashi (JP)

(72) Inventors: Tetsuo OYAGI, Yamanashi (JP); Hiroyuki OYAGI, Yamanashi (JP)

(21) Appl. No.: 19/434,738

(22) Filed: Dec. 29, 2025

(30) Foreign Application Priority Data
Feb. 15, 2025 (JP) 2025-022915

(51) Int. Cl. H04R 1/10; A61F 11/10

(52) U.S. Cl. CPC H04R 1/1083; A61F 11/10; H04R 1/1016

(57) ABSTRACT

Provided is an external auditory canal mounting device that not only has good sound insulating performance but also is easy to attach to and detach from an external auditory canal, has durability for repeated use, and is hygienic in that washing and the like can be easily performed. An external auditory canal mounting device to be inserted into and mounted in an external auditory canal includes: a columnar member formed of a tubular flexible member and insertable into the external auditory canal along an axial direction A of an axis extending from an opening portion of the external auditory canal toward an inside of the external auditory canal; and a protruding portion formed in a helical shape along the axial direction on an inner peripheral surface of the columnar member.

### TECHNICAL FIELD

The present invention relates to an external auditory canal mounting device, and more particularly, to an external auditory canal mounting device useful for application to earplugs that perform sound insulation in high-level noise environments and to ear tips and the like that conduct sound from an earphone into an external auditory canal favorably while blocking noise from the environments as far as possible.

### BACKGROUND ART

In cases such as performing sound insulation in high-level noise environments, an earplug is known as an instrument that is mounted in an external auditory canal to contribute to sound insulation. As one type of earplug according to the related art, a flange type earplug has been proposed (for example, refer to Patent Document 1). This type of flange type earplug includes a columnar main body and an umbrella-shaped flange portion that integrally extends in a radial direction from an outer peripheral surface of the main body, and is configured such that, by inserting the main body in an axial direction of an external auditory canal, an outer periphery of the flange portion is pressed against and brought into contact with an inner peripheral surface of the external auditory canal to block external noise.

In such a flange type earplug, at the time of mounting, a force is applied from the outside to push the earplug into the external auditory canal, and sound insulation is achieved by narrowing a space between the flange portion and the inner peripheral surface of the external auditory canal. At this time, a restoring force that tends to return the flange portion to an original shape constantly acts on the flange portion, and this restoring force acts as a force that pushes the flange type earplug outward from the external auditory canal and that tends to remove the flange type earplug from the ear. As a result, the flange type earplug in a state of being mounted in the external auditory canal becomes loose, thereby ultimately causing the earplug to come out of the external auditory canal. Therefore, in order to achieve stronger sound insulation, it is necessary to increase a pressing force by the flange portion; however, when a strong pressing force is applied, a force that tends to push the flange portion back also becomes relatively greater, making it difficult to maintain better mounting. In order to prevent this, the pressing force is limited to a level that can maintain the mounted state; accordingly, an achievable sound insulation level is limited, and, structurally, it is inherently difficult to achieve a high level of sound insulation.

However, since a soft material such as rubber or vinyl can be used as a molding material for the flange type earplug, molding is easy; in terms of characteristics of the material, it is less susceptible to the influence of moisture such as sweat and humidity; and, particularly, in a case of a material made of silicone, there is almost no deterioration due to external factors, durability is high, and long-term use is possible; this is a significant advantage.

As another type of earplug according to the related art, a foam type earplug has been proposed (for example, refer to Patent Document 2). This type of foam type earplug is formed by molding urethane foam, which is an elastic foamed polymer, into a columnar shape. Urethane foam is shaped by the expansion of small bubbles generated during formation, and has extensibility and flexibility, and has characteristics of deforming when compressed and slowly returning to the original shape when compression is released.

The foam type earplug molded from this urethane foam is inserted into the external auditory canal in a state in which the foamed material is compressed by fingers, utilizing deformability of the foam type earplug, and, as the foamed material tends to return to the original shape and expands in diameter until expansion is restricted by the shape of the external auditory canal, the foamed material conforms to the shape of the external auditory canal and seals the external auditory canal. Further, while the foam type earplug is mounted in the external auditory canal, the foam type earplug remains under a restriction on shape recovery, whereby stress is accumulated therein, and stress remaining therein constantly acts as an expanding force that presses against the external auditory canal to maintain and increase a sealing force.

Here, since the force pressing against the external auditory canal is oriented in a direction perpendicular to a tubular direction (axial direction) of the external auditory canal, the force is offset by 90 degrees with respect to an insertion direction of the foam type earplug into the external auditory canal, does not act as stress in a direction in which the foam type earplug is loosened or removed, and acts as residual stress pressing against the inner peripheral surface of the external auditory canal. That is, high sound insulating performance can be exhibited by a pressing force on the inner peripheral surface of the external auditory canal due to such residual stress.

However, polyurethane, which is a material of the foam type earplug, lacks stability in properties and has a drawback of being a material that readily deteriorates with age. In particular, it is susceptible to the influence of temperature and absorption of moisture or light (ultraviolet rays), and there is a possibility that the material changes occur due to long-term hydrolysis. As a result, under high humidity, an expansion (recovery) time becomes too short, making it difficult to mount appropriately.

In addition, due to deterioration of the material, it is not suitable for long-term use and there is a problem in durability. In particular, in a case of an earplug, since use is under a condition of being mounted in the external auditory canal, absorption of sweat cannot be avoided, and, when use starts, deterioration gradually starts. That is, deterioration starts together with the start of use, and, since functions decrease as a use period elapses, there is a possibility that a prescribed sound insulation amount is not satisfied.

Further, at the time of mounting the foam type earplug, since compressing the urethane foam with fingers and inserting the urethane foam into the external auditory canal is required, the operation is complicated under use conditions with frequent attachment and detachment. In addition, since absorption of moisture is a cause of deterioration, washing or alcohol disinfection of the foam type earplug is difficult once use has started; in applications in which hygiene is an issue, such as medical or food-related fields, this leads to a disposable usage form, thereby raising problems in terms of economic rationality.

As an external auditory canal mounting device of the same type, there is an ear tip. The ear tip includes a through-passage that penetrates between a proximal end face, which is an external-side end face of the earplug, and a distal end face (tympanic-membrane-side end face), which is an inner-side end face of the external auditory canal, and an opening portion on a proximal end face side is matched to a diameter and an outer shape on an earphone side, and is connected to a sound conducting portion from a speaker of the earphone, thereby constituting a so-called canal type earphone. In this type of canal type earphone, by inserting the ear tip into the external auditory canal, the canal type earphone is mounted in the external auditory canal. In this manner, the ear tip guides sound emitted from a speaker portion toward the tympanic membrane inside the external auditory canal through the through-passage of the ear tip and, by contact between an outer peripheral surface of the ear tip and the inner peripheral surface of the external auditory canal, blocks surrounding noise. That is, in the function of blocking surrounding noise except for sound emitted by the speaker, the ear tip exhibits the same function as the earplug.

### SUMMARY OF THE INVENTION

The present invention has been made in view of the related art described above, an object of the present invention is to provide an external auditory canal mounting device that not only has good sound insulating performance but also is easy to attach to and detach from an external auditory canal, has durability for repeated use, and is hygienic in that washing and the like can be easily performed.

According to a first aspect of the present invention for achieving the above-described object,

there is provided an external auditory canal mounting device including:

a columnar member configured to be inserted into and mounted in an external auditory canal along an axial direction of an axis extending from an inlet side of the external auditory canal toward an inside of the external auditory canal on a tympanic membrane side, the columnar member being a flexible member; and

a helical structure portion formed in a helical shape along the axial direction on the columnar member,

in which the columnar member is a tubular member with one end or both ends of the columnar member in the axial direction closed by a lid member, and

the helical structure portion is formed by a recessed portion or a protruding portion provided on an inner peripheral surface of the columnar member along the axial direction to a distal end portion that is an end portion on a side opposite to the inlet side.

According to a second aspect of the present invention,

in the external auditory canal mounting device described in the first aspect,

a tubular hollow portion of the columnar member is filled with an elastomer material.

According to a third aspect of the present invention,

in the external auditory canal mounting device described in the first aspect,

a through-hole that extends along the axial direction from an end portion of the columnar member on the inlet side to a distal end of the distal end portion is provided.

According to a fourth aspect of the present invention,

in the external auditory canal mounting device described in the second aspect,

a through-hole that extends along the axial direction from an end portion of the columnar member on the inlet side to a distal end of the distal end portion is provided.

According to a fifth aspect of the present invention,

in the external auditory canal mounting device described in the first aspect,

the columnar member incorporates conversion means for radiating sound vibrations converted and reproduced based on a predetermined electrical signal.

According to a sixth aspect of the present invention,

in the external auditory canal mounting device described in the second aspect,

the columnar member incorporates conversion means for radiating sound vibrations converted and reproduced based on a predetermined electrical signal.

According to a seventh aspect of the present invention,

in the external auditory canal mounting device described in the third aspect,

the columnar member incorporates conversion means for radiating sound vibrations converted and reproduced based on a predetermined electrical signal.

According to a eighth aspect of the present invention,

in the external auditory canal mounting device described in the fourth aspect,

the columnar member incorporates conversion means for radiating sound vibrations converted and reproduced based on a predetermined electrical signal.

According to the present invention, at the time of mounting the external auditory canal mounting device in the external auditory canal, in a case where the columnar member is inserted while applying stress acting in the axial direction of the columnar member, the helical structure portion extends in the axial direction of the columnar member by a spring effect. As a result, the columnar member is elongated along the axis of the external auditory canal. By being elongated in this way, in combination with a fact that the distal end portion of the columnar member has a smaller diameter than the diameter of the external auditory canal, the columnar member can be easily inserted into the external auditory canal. In this manner, the external auditory canal mounting device can be mounted by pressing an outer peripheral surface of the columnar member against the inner peripheral surface of the external auditory canal.

Here, the term "spring effect" as used in the present specification refers to the following property. When a deformable material is in a helical structure, in a case where a force is applied in a direction in which a helical pitch becomes greater, the outer diameter decreases and the helical pitch increases, and, as a result, an overall length is elongated. In addition, in a case where a force is applied in a direction in which the helical pitch becomes smaller, the outer diameter increases and the helical pitch decreases, and, as a result, the overall length is shortened. That is, extension and contraction occur back and forth in a helical direction and, together therewith, depending on conditions such as a thickness of the constituent material, a property of bending and of extending and contracting in non-fixed directions is exhibited.

In this case, the effect caused by the spring is such that, when the helical structure portion exhibiting the spring motion is pushed in as a columnar member having a diameter greater than the diameter of the external auditory canal, a force applied from an inner wall surface of the external auditory canal acts as stress in a direction in which the columnar member is narrowed. This stress compresses the helical structure portion from an outer peripheral side. As a result, the helical structure portion changes to a shape in which a diameter thereof decreases and the helical pitch increases.

Here, when the columnar member including the helical structure portion is held by fingers during mounting of the external auditory canal mounting device in the external auditory canal, the increased helical pitch cannot extend in an outward direction of the external auditory canal and elongation occurs only in a distal end direction (direction of the tympanic membrane).

Since the helical structure portion deformed by the above-described action tends to return to the original shape (original diameter), while being compressed (while mounted in the external auditory canal), the helical structure portion constantly generates expansive stress. As a result, due to stress pressing against the inner peripheral surface of the external auditory canal, the columnar member tends to assume a shape with as little gap as possible.

A human external auditory canal is not a regular tubular tube, and the external auditory canal is curved and has various inner diameters and shapes depending on positions in the canal, and is characterized by large individual differences. According to the present invention, since the helical structure portion changes an outer diameter and a direction of extension and contraction so as to conform at that position to inner diameters and shapes at positions in the individually different external auditory canal, as well as along the curvature (since the spring action has a property of bending and of extending and contracting in the non-fixed directions), a state with no gap (sealing) can be achieved throughout the entirety of the inner wall surface in contact. In other words, because of the property of the spring action, the helical structure portion deforms into a shape along the shape of the external auditory canal and is mounted and held in the external auditory canal in a state in which residual restoring stress that tends to restore the outer diameter to the original outer diameter remains. This residual restoring stress acts as compressive stress on an inner peripheral wall surface of the external auditory canal.

### Brief Description of the Drawings

FIGS. 1A to 1C are views showing an earplug which is an external auditory canal mounting device according to a first embodiment of the present invention, in which FIG. 1A is a perspective view thereof, FIG. 1B is a partially enlarged view showing an extracted and enlarged distal end portion thereof, and FIG. 1C is a longitudinal sectional view of FIG. 1A.

FIG. 2 is a perspective view showing a first modification example of the first embodiment.

FIGS. 3A and 3B are views showing a second modification example of the first embodiment, in which FIG. 3A is a perspective view thereof and FIG. 3B is a longitudinal sectional view thereof.

FIG. 4 is a perspective view showing a third modification example of the first embodiment of the present invention.

FIG. 5 is a perspective view showing a fourth modification example of the first embodiment of the present invention.

FIG. 6 is a perspective view showing a fifth modification example of the first embodiment of the present invention.

FIGS. 7A and 7B are views showing an earplug which is an external auditory canal mounting device according to a second embodiment of the present invention, in which FIG. 7A is a perspective view, and FIG. 7B is a longitudinal sectional view thereof.

FIGS. 8A and 8B are views showing an ear tip which is an external auditory canal mounting device according to a third embodiment of the present invention, in which FIG. 8A is a perspective view thereof, and FIG. 8B is a longitudinal sectional view thereof.

FIG. 9 is a perspective view in a case where an ear tip according to the third embodiment is connected to a canal type earphone.

FIG. 10 is a perspective view in a case where the ear tip according to the third embodiment is connected to an in-ear type earphone.

FIG. 11 is a perspective view in a case where the ear tip according to the third embodiment is connected to a stethoscope.

FIG. 12 is a perspective view showing a modification example of the third embodiment of the present invention.

### DETAILED DESCRIPTION OF THE INVENTION

### First Embodiment

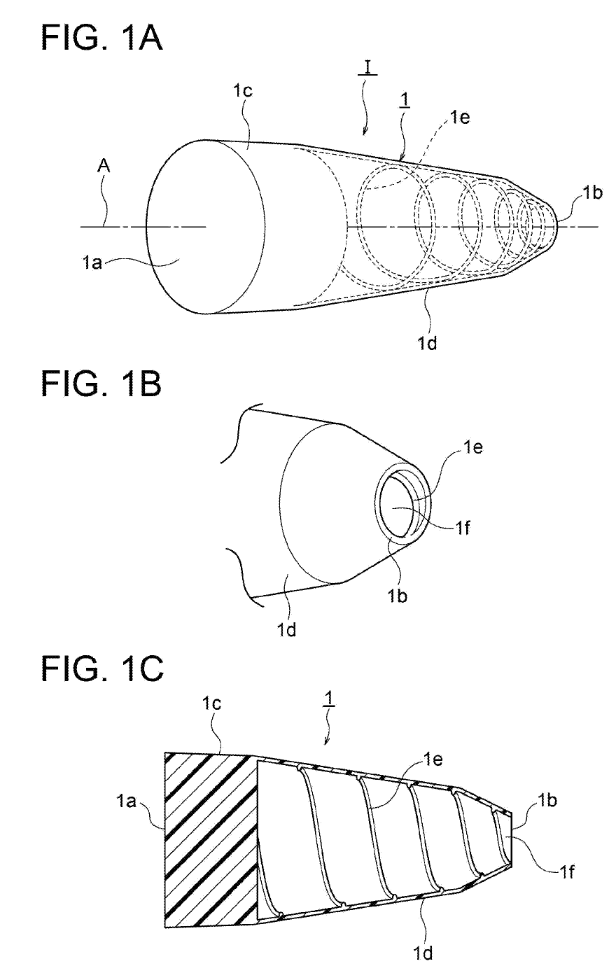

FIG. 1A is a perspective view showing an earplug which is an external auditory canal mounting device according to a first embodiment of the present invention, FIG. 1B is a partially enlarged view showing an extracted and enlarged distal end portion of the earplug, and FIG. 1C is a longitudinal sectional view of FIG. 1A. Although the earplug will be described in the present embodiment, the structure of an ear tip can be considered similarly, except that a through-passage that penetrates a space between a proximal end face, which is an end face on an inlet side of the external auditory canal (the same applies hereinafter), and a distal end face, which is an end face on a tympanic membrane side of the external auditory canal (the same applies hereinafter) is provided. That is, with respect to the function related to sound insulation, the ear tip exhibits the same function as the earplug.

As shown in FIGS. 1A and 1C, an earplug I according to the present embodiment includes a columnar member 1 formed of a flexible member and a protruding portion 1e which is a helical structure portion. The columnar member 1 is configured to be inserted into and mounted in the external auditory canal along an axial direction of an axis A extending from the inlet side of the external auditory canal toward an inside of the external auditory canal on the tympanic membrane side, and includes a distal end portion 1b that gradually decreases in diameter toward the tympanic membrane side to have a diameter smaller than a diameter of the external auditory canal.

The columnar member 1 in the present embodiment is a tubular member with the inlet side closed by a proximal end face 1a. Additionally, the helical structure portion is formed by the protruding portion 1e provided on an inner peripheral surface of the columnar member 1 along the direction of the axis A. That is, inside the tubular columnar member 1, the protruding portion 1e is formed in a helical shape along the direction of the axis A, and the protruding portion 1e constitutes the helical structure portion. Note that a similar helical structure can also be formed as a recessed portion.

In addition, with regard to the handedness of the helix, a left-hand (counterclockwise) helical structure is preferable for the right ear, and a right-hand (clockwise) helical structure is preferable for the left ear. This is because, owing to the shape characteristics of the external auditory canal, insertion into and mounting in the external auditory canal can be performed smoothly.

The earplug I in the present embodiment includes a tubular member including a solid portion 1c formed on a proximal end side from the proximal end face 1a toward the distal end portion 1b, and a hollow portion 1d formed on a distal end side continuously from the solid portion 1c. Although providing the solid portion 1c in this way is not essential, the following merits are obtained by providing the solid portion 1c. That is, at the time of inserting and mounting the earplug I into the external auditory canal, it is necessary to hold the earplug I with fingers and push it into the external auditory canal. By holding the solid portion 1c of the earplug I with fingers, a predetermined push-in force can be readily transmitted to the columnar member 1. Incidentally, in a case where the shape changes due to the holding force of the fingers, the push-in operation is hindered. That is, in a case where the earplug undergoes large deformation such as collapsing when held with fingers, the push-in operation is hindered. Therefore, a grip portion at this time is required to maintain a predetermined shape; to that end, at this position, the solid portion 1c, rather than a hollow portion, is required.

In addition, the outer shape of the columnar member 1 is generally circular; that is, the solid portion 1c has a circular column shape and the hollow portion 1d has a cylindrical shape; however, the outer shape is not limited to these. Depending on the shape of the external auditory canal, the cross section may be elliptical, and the like.

The hollow portion 1d gradually decreases in diameter toward the distal end so as to facilitate insertion into the external auditory canal. However, in this way, employing a shape in which the distal end portion has a reduced diameter is not an essential requirement. A cylindrical shape having a constant diameter may be employed. Additionally, a thickness of the hollow portion 1d may be formed thinner toward the distal end. This is for the following reason.

By varying the thickness of the material (for example, silicone) for molding the columnar member 1, thinner portions provide greater flexibility and can improve the mounting feel on the skin when mounted in the external auditory canal. On the other hand, since thicker portions are more rigid, transmission of the mounting operation is achieved. That is, by making the distal end softer and the grip portion (finger side) more rigid, a balance between ease of mounting and improved mounting feel can be achieved. This is because regions deeper in the external auditory canal are more sensitive. However, forming it in this way is also not an essential requirement.

As described above, on the inner peripheral surface of the hollow portion 1d, the helical protruding portion 1e (a recessed portion may be used) is formed along the axial direction from a proximal end face 1a side toward a distal end portion 1b side. The helical protruding portion 1e shown in FIG. 1A is single-start; however, the helical protruding portion 1e may be formed as a plurality of starts at predetermined intervals in the axial direction of the hollow portion 1d. Further, a structure in which a spring formed of plastic or the like is enclosed in the hollow portion 1d may also be employed. In short, the hollow portion 1d need only have a structure by which the above-described spring effect is obtained. That is, by virtue of the spring effect, when the earplug I is inserted into the external auditory canal, the helical structure generates a spring-like action along the curved shape of the external auditory canal and, at the same time, when a force is applied in a direction in which the helical pitch becomes greater, the outer diameter decreases and the helical pitch increases, whereby the overall length can be favorably elongated.

As a material for the earplug I, at least flexibility need only be provided, but silicone, which has durability, water resistance, and chemical resistance in addition to flexibility, is optimal. As other materials, rubber, vinyl, polyurethane, and the like can be favorably applied.

As shown in FIG. 1B, an opening portion 1f is provided at the distal end portion 1b so that air in the hollow portion 1d is discharged through the opening portion 1f.

Such an opening portion 1f may not always be necessary. In general, in a case where a volume of a space inside the columnar member 1 changes, a vent hole is required; however, since air itself is a compressible fluid, the volume change of the space can be absorbed depending on the amount of compression.

In addition, the distal end of the helical protruding portion 1e is connected to the thick distal end portion 1b of the opening portion 1f.

Since, according to the present embodiment, the earplug I having such a structure includes, on the inner peripheral surface of the hollow portion, the protruding or recessed portion forming the helical structure portion, the helical structure exhibits the spring effect along the curved shape of the external auditory canal when the earplug is inserted into the external auditory canal. That is, the earplug I, which is made of a soft material having flexibility and includes the helical protruding portion 1e on the inner peripheral surface, generates stress that tends to reduce the outer diameter and to become thinner as the outer peripheral surface of the earplug is pressed against the inner peripheral surface of the external auditory canal when inserted into the external auditory canal. At this time, since the protruding portion 1e, which is the helical structure portion on the inner peripheral surface, causes the stress to generate the spring effect in the protruding portion 1e, which is the helical structure portion, the pitch of the protruding portion 1e increases and the length of the tube increases, whereby the earplug I is elongated in the direction of the tympanic membrane that is a deep portion, while conforming to the curvature of the external auditory canal.

In the external auditory canal, from the opening portion toward the direction of the tympanic membrane, there are curved portions called a first curve and a second curve, and the distal end portion of the earplug I elongated by the spring effect has a property of readily bending in all directions under the influence of a coil shape of this spring structure. Therefore, the distal end portion readily bends along these curves of the external auditory canal, advances further in the direction of the tympanic membrane, and, at curved portions, bends at angles of the curves and comes to function like a key-shaped hook. This further enhances stability of mounting in the ear. Since such a shape change occurs so as to conform to the shape of each external auditory canal, the helical structure portion comes to function so as to freely deform and promptly adapt to an inherent shape of the external auditory canal of each individual.

At this time, the protruding portion 1e, which is the helical structure deformed by the spring effect, generates stress that tends to return the protruding portion to the original shape, whereby a restoring force that tends to expand acts constantly on the mounted earplug I in the contracted state. This restoring force that tends to expand acts as a force pressing against the inner peripheral surface of the external auditory canal, thereby firmly bringing the outer peripheral surface of the earplug I and the inner peripheral surface of the external auditory canal into tight contact with each other. Since the direction of the force due to such a restoring force is offset by about 90 degrees with respect to the insertion direction of the earplug I, which is the axial direction extending in the direction of the tympanic membrane of the external auditory canal, the stability of the mounted state of the earplug I is enhanced, and high sound insulation capability is exhibited.

Next, modification examples of the earplug having the basic structure shown in FIGS. 1A to 1C will be described. In each drawing, the same reference numerals are assigned to the same portions as those in FIGS. 1A to 1C, and duplicated descriptions will not be repeated.

### First Modification Example

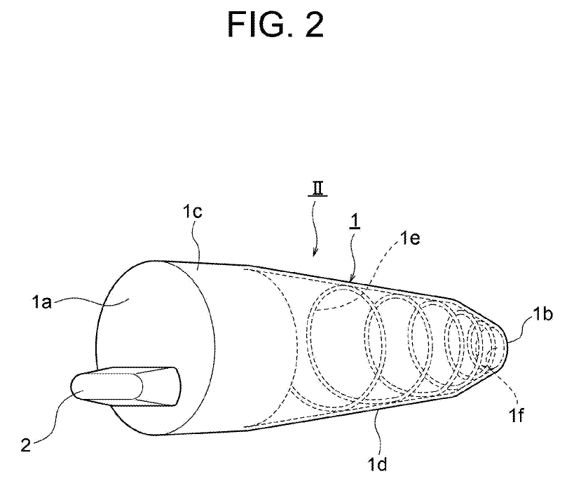

An earplug II shown in FIG. 2 is an earplug in which a knob 2 is attached to the proximal end face 1a. At attaching and detaching of the earplug II with respect to the external auditory canal, particularly at removal, by gripping the knob 2 and performing predetermined operations, efficiency of the operations can be improved.

### Second Modification Example

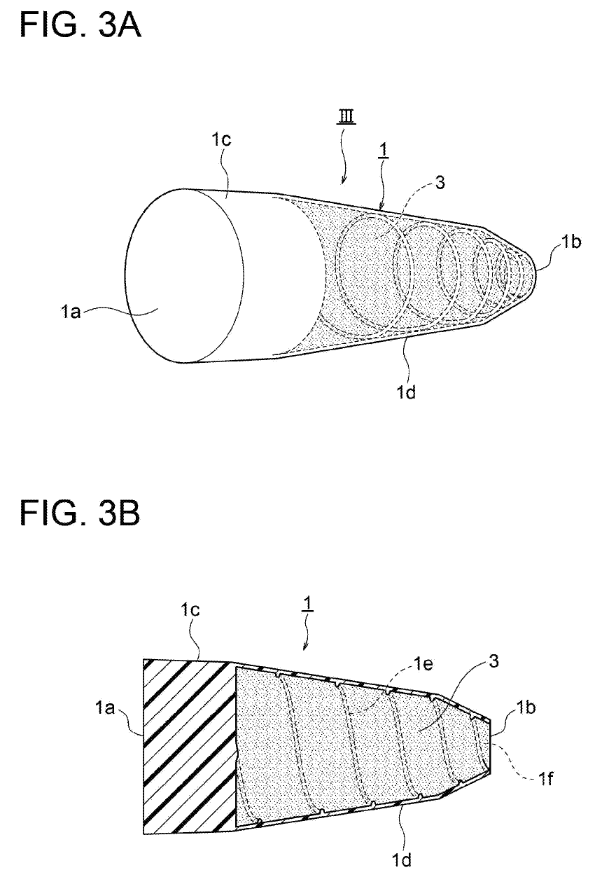

An earplug III shown in FIGS. 3A and 3B is an earplug in which the hollow portion 1d is filled with an elastomer material 3. In this example, by virtue of a function of the elastomer material 3 as a sound absorbing material, a more reliable sound insulation effect can be obtained.

### Third Modification Example

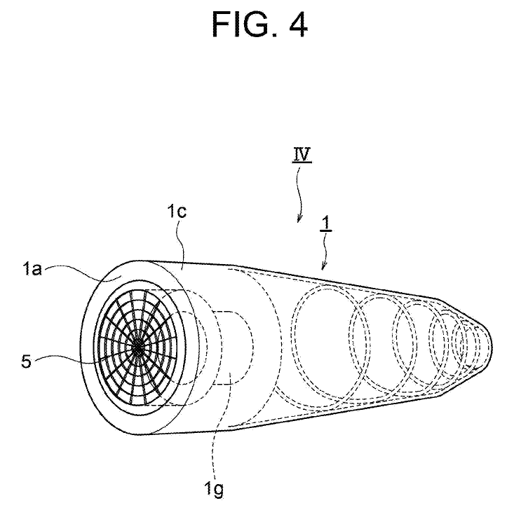

An earplug IV shown in FIG. 4 is an earplug in which an acoustic filter 5 (analog or digital) is fitted into a central portion of the proximal end face 1a so that sound of a specific frequency is selected or blocked, or is attenuated or amplified. More specifically, a sound conducting tube portion 1g that allows sound to pass between an external environment and the tympanic membrane is provided, and the acoustic filter 5, which is incorporated or removable and configured to adjust volume and sound quality, is fitted into one end thereof. By installing this acoustic filter 5, it is possible to adjust volume and sound quality transmitted when the earplug is used.

### Fourth Modification Example

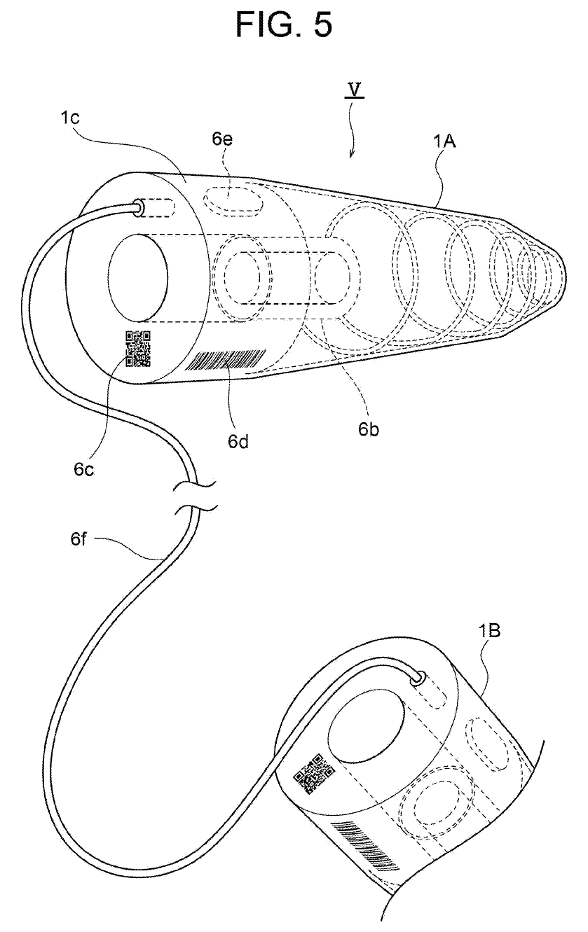

An earplug V shown in FIG. 5 is an earplug in which multifunctionality is pursued. For example, by embedding a metal member 6b in the solid portion 1c, even in a case where the earplug V is lost, mixing into food or the like can be readily detected by applying a metal detector, thereby preventing adverse effects in advance. In addition, by disposing a QR code 6c, a barcode 6d, a GPS chip 6e, and the like, acquisition of various types of information such as individual identification and whereabouts confirmation is facilitated. Further, by connecting a right-ear earplug 1A and a left-ear earplug 1B with a cord string 6f, loss due to dropping can be prevented in advance.

### Fifth Modification Example

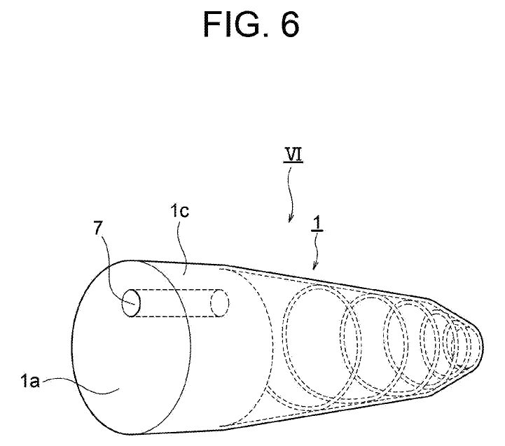

An earplug VI shown in FIG. 6 is an earplug in which a vent 7, which is a small hole, is provided in the solid portion 1c. By this vent 7, blocking performance and frequency characteristics can be adjusted.

### Second Embodiment

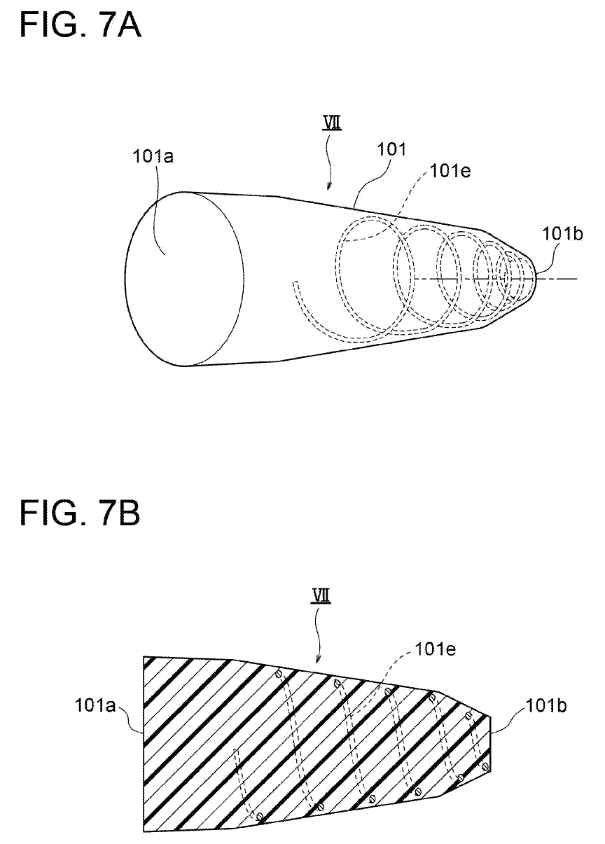

FIGS. 7A and 7B are views showing a second embodiment of the present invention, in which FIG. 7A is a perspective view thereof, and FIG. 7B is a longitudinal sectional view thereof. As shown in both drawings, in an earplug VII according to the present embodiment, a columnar member 101 is formed as a solid member. In this case, a material of the solid member uses a material capable of changing the shape with expansion and contraction, and a protruding portion 101e, which is a helical structure portion, is incorporated inside the columnar member 101 molded to be solid.

That is, in the present embodiment, the protruding portion 101e is present in elastomer, foamed urethane, or a polyolefin foam, which is a material that can be expanded and contracted. Therefore, the columnar member 101 need not be cylindrical, and need only be a member molded to be columnar without an internal space.

Here, the protruding portion 101e need not be of the same material as the columnar member 101 that is a solid member made of a flexible material, and may be molded from a flexible material that expands and contracts in a state in which a protruding portion 101e fabricated of another different material is incorporated. In addition, in a case where both are of the same material (a flexible material), the protruding portion 101e may have a greater hardness (hard state), and another portion, that is, the columnar member 101, may have a smaller hardness (soft state).

Since even silicone includes ultra-soft silicones having small hardness, it is similarly possible to fabricate such a structure by fabricating the protruding portion 101e from an appropriate material (either silicone having high hardness or a material such as another soft plastic) and molding the structure of the columnar member 101 in a state in which the protruding portion 101e is contained inside. Here, the spring effect is similarly exhibited in a case where the tubular columnar member 1 as in the first embodiment or the solid columnar member 101 as in the present embodiment is mounted in the external auditory canal.

### Third Embodiment

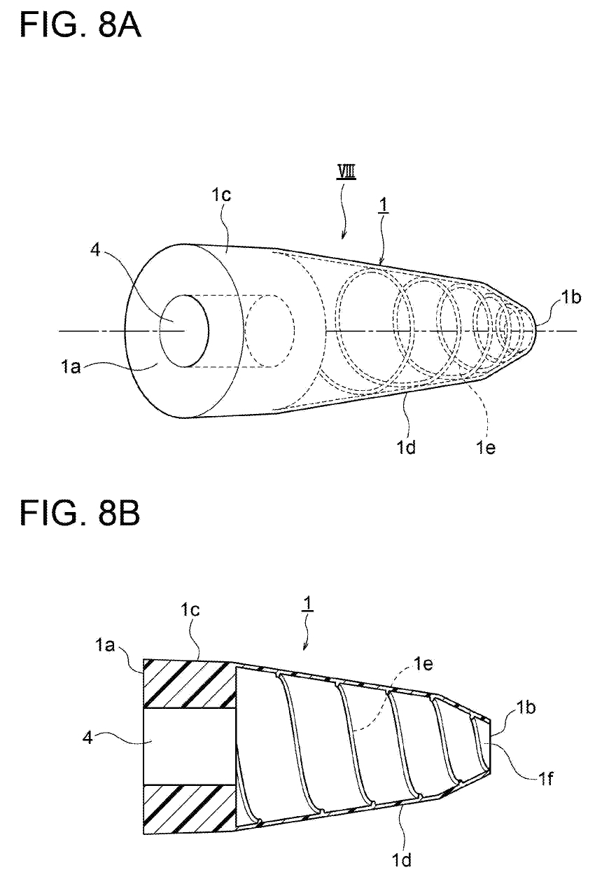

FIGS. 8A and 8B are views showing an ear tip which is an external auditory canal mounting device according to a third embodiment of the present invention, in which FIG. 8A is a perspective view thereof, and FIG. 8B is a longitudinal sectional view thereof. Here, the ear tip, in contrast to the earplugs described in the first and second embodiments that perform sound insulation in high-level noise environments, serves to block noise from the environments as far as possible and conduct sound from the earphone, hearing aids and sound amplifiers and so on into the external auditory canal favorably.

Therefore, as shown in FIGS. 8A and 8B, an ear tip VIII according to the present embodiment has a structure including a sound conducting hole 4 that is a through-hole that extends along the axial direction of the columnar member 1 from the proximal end face 1a, which is an end portion of the earplug I shown in FIGS. 1A to 1C on the inlet side of the columnar member 1, to the distal end portion 1b. The ear tip VIII is the same as the earplug I shown in FIGS. 1A to 1C except that the sound conducting hole 4 is provided. Therefore, in FIGS. 8A and 8B, the same reference numerals are assigned to the same portions as those in FIGS. 1A to 1C, and duplicated descriptions will not be repeated.

### Application Examples

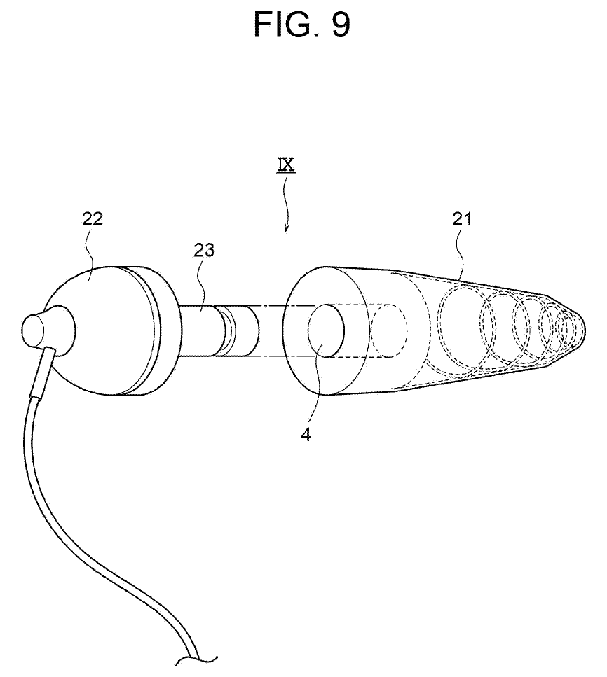

Application examples of the ear tip according to the third embodiment as described above will be described here. FIG. 9 is a perspective view in a case where an ear tip 21 according to the third embodiment is connected to a canal type earphoneIX. As shown in FIG. 9, in this case, the ear tip 21 and the canal type earphone IX are integrated by inserting a sound conducting tube 23, which is formed so as to protrude from a main body 22 that incorporates a speaker portion of the canal type earphoneIX, into the sound conducting hole 4 of the ear tip 21.

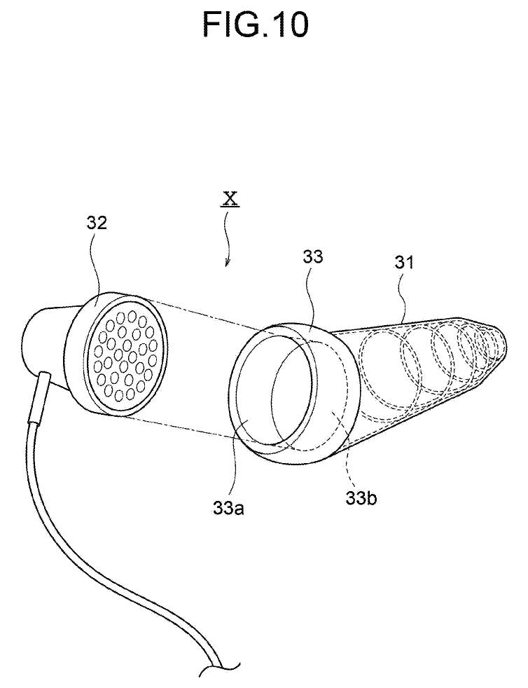

FIG. 10 is a perspective view in a case where an ear tip 31 according to the third embodiment is connected to an in-ear type earphone X. As shown in FIG. 10, in this case, a main body 32 incorporating a speaker of the in-ear type earphoneX is integrated with an ear tip 31 via a connecting/sound conducting tube 33. In other words, the main body 32 is fitted into the connecting/sound guide tube 33 via one opening portion 33a of the connecting/sound guide tube 33, and the base end of the ear tip 31 is fitted into the connecting/sound guide tube 33 via an opening portion 33b on the opposite side of the connecting/sound guide tube 33.

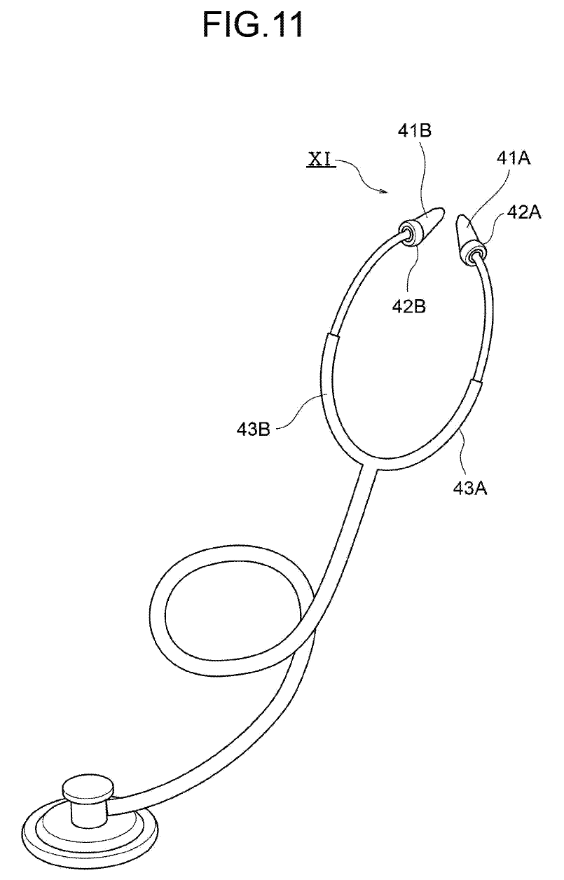

FIG. 11 is a perspective view in a case where ear tips 41A and 41B according to the third embodiment are connected to a stethoscope. As shown in FIG. 11, in this case, the ear tips 41A and 41B mounted in, for example, the left and right ears of a physician are integrated via adapters 42A and 42B having a sound conducting function that are connected to left and right tubes 43A and 43B of a stethoscope XI.

### Modification Example

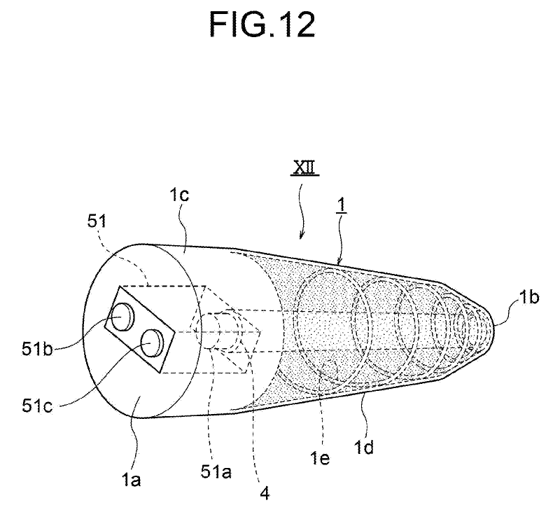

A modification example relating to the third embodiment shown in FIGS. 8A and 8B will be described here with reference to FIG. 12. In the solid portion 1c of the columnar member 1 in an ear tip XII of the modification example shown in FIG. 12, a speaker 51 is embedded. The speaker 51 is a core component of the earphone that is connected to a sound source in a wired or wireless manner, converts an electrical signal sent from the sound source into sound, conducts this reproduced sound into the external auditory canal via the sound conducting hole 4, and further guides the reproduced sound to the tympanic membrane. The speaker 51 includes a diaphragm 51a that radiates the sound reproduced by converting the electrical signal toward the sound conducting hole 4 as air vibration, and connection terminals (+, −) 51b and 51c for receiving a predetermined electrical signal.

In the earphone including the ear tipXII according to the present modification example, a good sound insulation effect based on the spring effect exhibited by the earplug I can be obtained. As a result, the earphone allows desired sound to be heard favorably while blocking external noise, without allowing the sound reproduced by the speaker 51 to leak to the outside as far as possible.

As described above, the structural differences between the earplugs I to VII and the ear tipsVIII andXII reside only in that the former do not include the sound conducting hole 4, whereas the latter have the sound conducting hole 4. Accordingly, with regard to sound insulation, the ear tip also exhibits the same function as the earplug.

As a result, in a case where the sound conducting hole 4 is formed in the earplugsII to VII according to the modification examples shown in FIGS. 2 to 6 and the second embodiment shown in FIG. 7, it is possible to form ear tips corresponding to the modification examples and the second embodiment. Moreover, even in the modification example related to the third embodiment shown in FIG. 12, by applying a so-called bone conduction speaker, which transmits vibration directly to the skull as solid propagating sound without mediation of air vibration, instead of a method of transmitting sound waves to the tympanic membrane as air vibration, it is possible, without forming the sound conducting hole 4, to make the earplugs II to VII according to the first to fifth modification examples and the second embodiment function as an earpiece similar to the ear tipVIII.

It should be noted that, although the columnar members 1 and 101 in the embodiments and modification examples are formed to have a shape that gradually decreases in diameter toward the distal end portion 1b, the present invention is not limited to this. As long as the columnar members 1 and 101 are flexible members formed so as to be insertable into and mountable in the external auditory canal along the axial direction of the axis A extending from the inlet side toward the inside of the external auditory canal on the tympanic membrane side, further limitations are not necessarily required. In addition, the protruding portions 1e and 101e may be formed on outer peripheral surfaces of the columnar members 1 and 101.

With regard to the earplugs, although the earplugs I to VII mainly intended for a soundproofing effect have been mainly described, the earplugs are also usable as earplugs intended for waterproofing, such as for swimming. Further, use as an earplug intended for heat retention to prevent stenosis of the external auditory canal, which is called so-called surfer's ear, is also conceivable.

1. An external auditory canal mounting device comprising:
a columnar member configured to be inserted into and mounted in an external auditory canal along an axial direction of an axis extending from an inlet side of the external auditory canal toward an inside of the external auditory canal on a tympanic membrane side, the columnar member being a flexible member; and
a helical structure portion formed in a helical shape along the axial direction on the columnar member,
wherein the columnar member is a tubular member with one end or both ends of the columnar member in the axial direction closed by a lid member, and
the helical structure portion is formed by a recessed portion or a protruding portion provided on an inner peripheral surface of the columnar member along the axial direction to a distal end portion that is an end portion on a side opposite to the inlet side.

2. The external auditory canal mounting device according to claim 1,
wherein a tubular hollow portion of the columnar member is filled with an elastomer material.

3. The external auditory canal mounting device according to claim 1,
wherein a through-hole that extends along the axial direction from an end portion of the columnar member on the inlet side to a distal end of the distal end portion is provided.

4. The external auditory canal mounting device according to claim 2,
wherein a through-hole that extends along the axial direction from an end portion of the columnar member on the inlet side to a distal end of the distal end portion is provided.

5. The external auditory canal mounting device according to claim 1,
wherein the columnar member incorporates conversion means for radiating sound vibrations converted and reproduced based on a predetermined electrical signal.

6. The external auditory canal mounting device according to claim 2,
wherein the columnar member incorporates conversion means for radiating sound vibrations converted and reproduced based on a predetermined electrical signal.

7. The external auditory canal mounting device according to claim 3,
wherein the columnar member incorporates conversion means for radiating sound vibrations converted and reproduced based on a predetermined electrical signal.

8. The external auditory canal mounting device according to claim 4,
wherein the columnar member incorporates conversion means for radiating sound vibrations converted and reproduced based on a predetermined electrical signal.
